(19) United States
(12) Patent Application Publication
(10) Pub. No.: US 2026/0232646 A1
(43) Pub. Date: Aug. 13, 2026

(54) METHOD FOR TREATING POSTOPERATIVE PAIN AND USE THEREOF

(71) Applicant: ZHEJIANG CUIZE PHARMACEUTICAL TECHNOLOGY CO., LTD., Hangzhou (CN)

(72) Inventors: Siping SUN, Hangzhou (CN); Dongjie FENG, Hangzhou (CN); Yu YAO, Hangzhou (CN); Long LI, Hangzhou (CN); Ruichen LI, Hangzhou (CN)

(73) Assignee: ZHEJIANG CUIZE PHARMACEUTICAL TECHNOLOGY CO., LTD., Hangzhou (CN)

(21) Appl. No.: 19/278,846

(22) Filed: Jul. 24, 2025

(30) Foreign Application Priority Data
Feb. 11, 2025 (CN) 202510152048.2

(51) Int. Cl. A61K 31/445; A61K 9/00; A61K 9/10; A61P 25/04

(52) U.S. Cl. CPC A61K 31/445; A61K 9/0019; A61K 9/10; A61P 25/04

(57) ABSTRACT

A method for treating post-hemorrhoidectomy pain and use thereof are provided. The method includes administering a local anesthetic to a patient in need thereof. The local anesthetic includes a pharmaceutical composition including ropivacaine crystal A. The ropivacaine crystal A has an aspect ratio of about 1:(1-2), a D₅₀ of about 5-10 μm, and a crystal particle size distribution width value of about 1-2.

### CROSS REFERENCE TO THE RELATED APPLICATIONS

This application is based upon and claims priority to Chinese Patent Application No. 202510152048.2, filed on Feb. 11, 2025, the entire contents of which are incorporated herein by reference.

### TECHNICAL FIELD

The present disclosure relates to the field of biomedicine and, in particular, to a method for treating postoperative pain and use thereof.

### BACKGROUND

Hemorrhoids are one of the most common anorectal diseases in clinical practice. Hemorrhoids can be divided into internal hemorrhoids, external hemorrhoids, and mixed hemorrhoids according to the site of disease. The treatment method for hemorrhoids (piles) is mainly selected according to its severity, symptoms, and the specific conditions of patients. Generally, it can be divided into two categories: non-surgical treatment and surgical treatment. Surgical treatment is often considered when the hemorrhoid progresses to a severe stage (e.g. degree III or IV) or when non-surgical therapy is ineffective. Common surgical methods include: hemorrhoidectomy, stapled hemorrhoidopexy, hemorrhoidal artery ligation (HAL), sclerosing agent injection therapy, and rubber band ligation.

Postoperative pain is a common complication of hemorrhoid surgery. Post-hemorrhoidectomy pain is mostly acute pain, which is a normal physiological response to tissue demage or injury. It has physiological significance, and can be used as a warning of risk or injury. If this pain is not timely controlled, it will not only have a significant impact on the psychological and physiological functions of the patients, such as anxiety, insomnia, dysphoria, nausea, vomiting, urinary retention, etc. and postoperative acute pain may develop into chronic pain, lasting for half a year or even decades, or even lead to disability. Postoperative pain usually begins to develop after the surgery or after the anesthetic effect disappears, and generally lasts for 3-7 days, of which the first 3 days are the most intense pain and also the critical period of postoperative analgesia. Clinically, it is generally alleviated by oral analgesics, external anesthetic ointment, warm water baths, and other measures.

Currently, the analgesic drugs in clinical use include: opioid analgesics, such as: morphine, buprenorphine, tramadol, fentanyl, sufentanil, alfentanil, remifentanil and the like. Such drugs have a rapid onset of action and strong analgesic effects, but the maintenance time is short, and side effects are large. Representative drugs are ropivacaine, lidocaine, bupivacaine, levobupivacaine, procaine, tetracaine, etc. Non-steroidal anti-inflammatory drugs are divided into two categories: Drugs for oral administration and injection. Oral drugs mainly include ibuprofen, meloxicam, celecoxib, lornoxicam, diclofenac, etc. The main drugs for injection include parecoxib, lornoxicam, flurbiprofen axetil, and ketorolac, etc. These drugs are mainly used for analgesia after small and medium-sized surgery. However, for the postoperative pain of major surgery, the analgesic effect of this drug is not obvious, and it needs to be combined with other analgesics (opioids or local anesthetics) to achieve the effect. In order to lessen the pain of patients after hemorrhoid surgery, stabilize the functions of various organs of patients, improve patient prognosis, and shorten patient stay, analgesia for injurious pain, especially long-term analgesia, has always been an urgent issue to be solved in clinical practice.

### SUMMARY

One objective of the present disclosure, the present disclosure provides a method for treating or preventing postoperative pain including administering to an individual in need thereof a pharmaceutical composition including ropivacaine crystal A.

In one embodiment of the present disclosure, the present disclosure also provides a pharmaceutical composition for treating or preventing postoperative pain. The pharmaceutical composition includes ropivacaine crystal A.

In some embodiments, the ropivacaine crystal A has an aspect ratio of about 1:1-2.

In some embodiments, at least about 80%, at least about 85%, at least about 90%, and at least about 95% of the crystals in the ropivacaine crystal A have an aspect ratio of about 1:(1~2).

In some embodiments, the ropivacaine crystal A has a D₅₀ of about 5-10 μm.

In some embodiments, the ropivacaine crystal A has a crystal particle size distribution width (SPAN) value of less than 3.

In some embodiments, at least 95% of the ropivacaine crystal A have a crystal particle size distribution width (SPAN) value between about 1-2.

In some embodiments, at least 80%, 85%, 90%, and 95% of the ropivacaine crystal A have a crystal particle size distribution width (SPAN) value between about 1-2.

In some embodiments, the ropivacaine crystal A has the particle morphology as shown in FIG. 2.

In some embodiments, the ropivacaine crystal A has XRPD diffraction peaks as shown in FIG. 1.

In one embodiment of the present disclosure, the methods of the present disclosure are used to treat or prevent pain resulting from post-hemorrhoid surgery.

In some embodiments, postoperative pain includes traumatic pain, chemical pain, neuropathic pain, and inflammatory pain.

In some embodiments, postoperative pain includes acute pain and/or chronic pain.

In some embodiments, treating or preventing includes attenuating, inhibiting, reversing, slowing, or stopping the progression or severity of an existing condition, disease, disorder, or symptom.

In some embodiments, treating or preventing includes curing, alleviating, lessening, or eliminating the progression or severity of a disease, condition, disorder, or symptom.

In one embodiment of the present disclosure, the ropivacaine crystal A is administered to an individual in need thereof by injection.

In some embodiments, the ropivacaine crystal A is administered to an individual in need thereof by perianal injection.

In some embodiments, the methods of the present disclosure include topically administering the pharmaceutical composition to the perianal region.

In some embodiments, the methods of the present disclosure include administering the pharmaceutical composition to the anal verge site.

In some embodiments, the methods of the present disclosure include administering the pharmaceutical composition to the perianal peripheral nerve site.

In some embodiments, the methods of the present disclosure include applying the pharmaceutical composition 0.5-3 cm from the anal verge.

In some embodiments, the methods of the present disclosure include injecting the pharmaceutical composition into the perianal anal sphincter and subcutaneous tissue.

In one embodiment of the present disclosure, there is provided a method for injectable administration of a pharmaceutical composition for postoperative analgesia to an individual in need thereof, including:
selecting an entry point of the injection needle at a body part of the patient, including selecting a needle entry point and a direction of the injection needle;
inserting an injection needle into a patient at an entry point;
topically administering a pharmaceutical composition to the body of an individual in need thereof via an injection needle. The pharmaceutical composition may include physiological saline and an active drug, e.g. the ropivacaine crystal A.

In some embodiments, the entry point of the injection needle is selected to be at least 4 sites, such as 4-8 sites preferably 4, 5, 6, 7, or 8 sites.

In some embodiments, the 4 sites are at 3 o'clock, 6 o'clock, 9 o'clock, and 12 o'clock of lithotomy position, respectively.

In one embodiment of the present disclosure, the present disclosure provides a method for administering a pharmaceutical composition for postoperative analgesia to a perianal peripheral nerve of an individual in need thereof, including: selecting an entry point of the injection needle around the anus of an individual in need thereof, such as a human patient; inserting the injection needle into the body of an individual in need thereof from a selected site;

selecting at least 4 sites for the entry point of the injection needle; for example, 4-8 sites, preferably 4, 5, 6, 7, or 8 sites.

In some embodiments, the 4 sites are at 3 o'clock, 6 o'clock, 9 o'clock, and 12 o'clock of lithotomy position, respectively.

A therapeutically effective amount of the pharmaceutical composition is topically injected around the anus of an individual in need thereof via an injection needle; wherein the pharmaceutical composition includes the ropivacaine crystal A. Prior to use, the pharmaceutical compositions may be prepared in an injectable form using sterile water for injection or saline. Among them, free ropivacaine is used as an amide local anesthetic to inhibit the impulse conduction of peripheral sensory nerves and provide postoperative analgesia to patients.

In one embodiment of the present disclosure, the present disclosure provides a method for perianal administration of a pharmaceutical composition for postoperative analgesia to a human patient, including:

selecting the needle entry point of the injection needle around the patient's anus.

The needle tip of the injection needle is advanced to the anal sphincter along a trajectory from the needle entry point to the plane of the patient's sphincter.

A therapeutically effective amount of the pharmaceutical composition is administered to the localized area through an injection needle while the needle is withdrawn. Wherein the pharmaceutical composition includes the ropivacaine crystal A, whereby a pharmaceutical composition for postoperative analgesia is administered to the perianal region of a human patient.

In one embodiment of the present disclosure, there is a method for treating post-hemorrhoidectomy pain in a patient, including:
selecting an entry point of the injection needle around the anus of an individual in need thereof;
selecting at least 4 sites; for example, 4-8 sites, preferably 4, 5, 6, 7, or 8 sites.
inserting the injection needle into the body of a patient from a selected site;
topically injecting a physiological saline and a therapeutically effective amount of the pharmaceutical composition around the anus of an individual in need thereof via an injection needle; wherein the pharmaceutical composition includes the ropivacaine crystal A.

In one embodiment of the present disclosure, there is provided a method for performing local infiltration of a perianal local anesthetic to lessen pain after a hemorrhoidectomy in an individual in need thereof, including:
selecting an entry point of the injection needle around the anus of an individual in need thereof;
selecting at least 4 sites, wherein the injection points include 0.5-3 cm away from the anal verge, at 3 o'clock, 6 o'clock, 9 o'clock, and 12 o'clock of the lithotomy position;
inserting the injection needle into the body of an individual in need thereof from a selected site;
topically injecting a physiological saline and a therapeutically effective amount of the pharmaceutical composition around the anus of an individual in need thereof via an injection needle; wherein the pharmaceutical composition includes the ropivacaine crystal A.

In some embodiments, the methods of the present disclosure include administering the pharmaceutical composition to a patient by local infiltration around the anus.

In some embodiments, the methods of the present disclosure include administering the pharmaceutical composition to the anal verge site.

In some embodiments, the methods of the present disclosure include administering the pharmaceutical composition 0.5-3 cm from the anal verge and injecting the pharmaceutical composition at at least 4 sites selected.

In some embodiments, the injection needle is a 21G needle, or other conventional injection needle of similar gauge that can be used for topical administration perianal, the length of the needle being between 30 mm and 40 mm.

In some embodiments, the injection needle is non-occlusive.

In some embodiments, the injection needle is inserted around the anus of an individual in need thereof to a depth of greater than 3 cm.

In some embodiments, the pharmaceutical composition is administered to the perianal local infiltration.

In some embodiments, the pharmaceutical composition is injected at at least 4 sites selected 0.5-3 cm from the anal verge.

In some embodiments, when the patient is in the lithotomy position, 4 mL of the pharmaceutical composition is injected between the subcutaneous and anal sphincter plane using a 21 G needle vertically inserted at 6 o'clock direction 0.5-3 cm from the anal verge by withdrawing the needle.

In some embodiments, when the patient is in the lithotomy position, 4 mL of the pharmaceutical composition is injected between the subcutaneous and anal sphincter plane using a 21 G needle vertically inserted at 12 o'clock direction 0.5-3 cm from the anal verge by withdrawing the needle.

In some embodiments, when the patient is in the lithotomy position, 2 mL of the pharmaceutical composition is injected between the subcutaneous and anal sphincter plane using a 21 G needle vertically inserted at 3 o'clock direction 0.5-3 cm from the anal verge.

In some embodiments, when the patient is in the lithotomy position, 2 mL of the pharmaceutical composition is injected between the subcutaneous and anal sphincter plane using a 21 G needle inserted 45° downward at 3 o'clock direction 0.5-3 cm from the anal verge.

In some embodiments, when the patient is in the lithotomy position, 2 mL of the pharmaceutical composition is injected between the subcutaneous and anal sphincter plane using a 21 G needle inserted 45° upward at 3 o'clock direction 0.5-3 cm from the anal verge.

In some embodiments, when the patient is in the lithotomy position, 2 mL of the pharmaceutical composition is injected between the subcutaneous and anal sphincter plane using a 21 G needle vertically inserted at 9 o'clock direction 0.5-3 cm from the anal verge.

In some embodiments, when the patient is in the lithotomy position, 2 mL of the pharmaceutical composition is injected between the subcutaneous and anal sphincter plane using a 21 G needle inserted 45° downward at 9 o'clock direction 0.5-3 cm from the anal verge.

In some embodiments, when the patient is in the lithotomy position, 2 mL of the pharmaceutical composition is injected between the subcutaneous and anal sphincter plane using a 21 G needle inserted 45° upward at 9 o'clock direction 0.5-3 cm from the anal verge.

In some embodiments, 10 mL of the pharmaceutical composition is injected as a local infiltrate into the anal sphincter and subcutaneous tissue using a 21 G needle at the margin of the surgical incision.

In some embodiments of the present disclosure, the pharmaceutical composition is administered by injection.

In some embodiments of the present disclosure, the pharmaceutical composition refers to a composition in a form suitable for administration. Suitable forms of administration depend on the use or route of entry (including, but not limited to, subcutaneous injection), single injection, continuous injection, indwelling catheters, and continuous infusion.

In some embodiments of the present disclosure, the amount of the pharmaceutical composition in a single injection does not exceed 6 mL, preferably 5 mL, 4 mL, 3 mL, 2 mL.

In some embodiments of the present disclosure, the method for treating or preventing postoperative pain includes injecting about 30 mL of the pharmaceutical composition into an individual in need thereof.

In some embodiments of the present disclosure, the pharmaceutical composition is a liquid composition, an emulsion, a suspension, or a dry formulation.

In some embodiments of the present disclosure, the pharmaceutical composition further includes any one selected from the group consisting of a thickener, a surfactant, a filler, or any combination thereof.

In some embodiments of the present disclosure, the therapeutic effect of a high dose of the ropivacaine crystal A exceeds the therapeutic effect of a low dose of the ropivacaine crystal A.

In some embodiments of the present disclosure, the lower dose is between 200 mg-400 mg.

In some embodiments of the present disclosure, the higher dose is between 400 mg-600 g.

In some embodiments of the present disclosure, the dosage may be selected between 200 mg-800 mg, preferably 200 mg-750 mg, 200 mg-700 mg, 200 mg-650 mg, 200 mg-600 mg, 200 mg-550 mg, 200 mg-500 mg, 200 mg-450 mg, 200 mg-400 mg, 200 mg-350 mg, 200 mg-300 mg, 200 mg-250 mg, 350 mg-650 mg, 400 mg-600 mg, 450 mg-550 mg, and 500 mg-600 mg.

In one embodiment of the present disclosure, the pharmaceutical composition for preventing or treating postoperative pain may be used in conjunction with other anesthetics or other analgesics, or in an anesthetic or analgesic regimen with other anesthetics or other analgesics. The pharmaceutical composition includes the ropivacaine crystal A.

Examples of anesthetic agents include, but are not limited to: propofol, etomidate, thiopental sodium, midazolam, diazepam, ketamine, benzocaine, chloroprocaine, cocaine, cyclomethycaine, dimethocaine, propoxycaine, procaine, proparacaine, tetracaine, articaine, bupivacaine, carticaine, dibucaine, etidocaine, levobupivacaine, lidocaine, mepivacaine, piperocaine, prilocaine, ropivacaine, trimecaine, and tetrodotoxin. Examples of amide anesthetics include, but are not limited to, articaine, bupivacaine, carticaine, dibucaine, etidocaine, levobupivacaine, lidocaine, mepivacaine, piperocaine, prilocaine, ropivacaine, and trimecaine.

Examples of analgesics may include opioid analgesics and non-opioid analgesics. Examples of opioid analgesics include hydrocodone, oxycodone, propoxyphene or fentanyl, thiocarbamate, p-nitrobenzoylhydrazone, o-methyloxime, hemicarbazone, or bis(methylcarbamate), pharmaceutically acceptable salt of oxycodone or its thiocarbamoyl ketons, p-nitrobenzhydrazone, biuret, or bis-methylcarbamate. Examples of non-opioid analgesics include aspirin, acetaminophen, NSAIDs, gabapentin, a COX-2 inhibitor, a topical analgesic, an antidepressant, an atypical analgesic, a psychotropic drug, an NMDA receptor antagonist, an adrenergic receptor antagonist, and a synthetic drug with anesthetic properties.

Optionally, the pharmaceutical composition for preventing or treating postoperative pain may be used in conjunction with other anesthetics, an anesthesia adjuvant, or other analgesics. Wherein, the other anesthetics include general anesthetics and local anesthetics, the general anesthetics include etomidate, ketamine, ciprofol, propofol, and thiopental sodium; and the local anesthetics are selected from benzocaine, chloroprocaine, cocaine, cyclomethycaine, dimethocaine, propoxycaine, procaine, amethocaine, articaine, bupivacaine, etidocaine, levobupivacaine, lidocaine, mepivacaine, and trimecaine;
and/or the anesthesia adjuvants include midazolam and diazepam;
and/or the other analgesics include opioid analgesics, antipyretic analgesic anti-inflammatory drugs, and adjuvant analgesics;
wherein, the opioid analgesics are selected from hydrocodone, dextropropoxyphene, codeine, dihydrocodeine, tramadol, morphine, oxycodone, hydromorphone, fentanyl, sufentanil, remifentanil, buprenorphine, pethidine (dolantin), nalbuphine, methadone, levorphanol, pentazocine, or naloxone; the antipyretic analgesics are selected from: acetaminophen and NSAIDs; wherein the NSAIDs are selective COX-2 inhibitors such as celecoxib, or non-selective inhibitors such as ibuprofen, aspirin, diclofenac, and naproxen; the adjuvant analgesics are selected from: anticonvulsants such as gabapentin, tricyclic antidepressants such as amitriptyline, psychotropic drugs, NMDA receptor antagonists such as ketamine, α₂-adrenergic receptor agonists such as dexmedetomidine, and glucocorticoids such as dexamethasone.

In some embodiments of the present disclosure, the ropivacaine crystal A is administered simultaneously, sequentially, or alternately with other anesthetics or analgesics.

In one embodiment of the present disclosure, there is also provided a process for preparing the ropivacaine crystal A. The method includes:

Ropivacaine hydrochloride is dissolved in the solvent and stirred at normal temperature for dissolution. After complete dissolution, sodium hydroxide solution is added to adjust pH to alkaline, then heated the drug solution to 90° C.-100° C., sheared, and dispersed while heating, and the temperature is kept for more than 20 minutes. Filtration is performed to give the target crystal. The solvent may be water. The reaction temperature of the dissolution step is less than 40° C., preferably room temperature. The step of adjusting the pH refers to adjusting the pH to a value greater than 7.

In some embodiments, the ropivacaine crystal A prepared in accordance with the present disclosure is tested to have a crystal particle size distribution width (SPAN) value of less than 3.

In some embodiments, at least 95% of the crystals in the ropivacaine crystal A prepared according to the present disclosure have a crystal particle size distribution width (SPAN) value of about 1-2 as measured.

In some embodiments, at least 80%, 85%, 90%, and 95% of the crystals in the ropivacaine crystal A prepared according to the present disclosure are tested to have a crystal particle size distribution width (SPAN) value of about 1-2.

In some embodiments, the ropivacaine crystal A prepared according to the present disclosure has been tested to have an aspect ratio of about 1:1-2.

In some embodiments, the ropivacaine crystal A prepared by the present disclosure has XRPD diffraction peaks at 8.894, 10.485, 13.045, 13.580, 14.300, 16.348, 17.918, 19.122, 19.470, 19.984, 20.315, 21.107, 22.567, 23.529, 24.459, 25.593, 26.309, 31.295, 31.880, 32.449, as determined.

In some embodiments, the ropivacaine crystal A prepared by the present disclosure has been detected to have XRPD diffraction peaks as shown in FIG. 1.

In one embodiment of the present disclosure, the present disclosure also provides the use of the ropivacaine crystal A in the manufacture of a medicament for the treatment of postoperative pain.

In one embodiment, the postoperative pain includes postoperative pain for hemorrhoid treatment.

In one embodiment, the medicament is administered to the patient by local injection.

In one embodiment, the drug is administered to the patient by local infiltration. The local infiltration has all of the characteristics set forth in the foregoing treatment methods.

In one embodiment, the medicament may be prepared as any pharmaceutical composition suitable for administration by injection. The pharmaceutical compositions have all of the features set forth in the foregoing methods of treatment.

By applying the ropivacaine crystal A of the present disclosure in the form of a perianal local infiltration, a long-lasting analgesic effect can be produced, showing a significant advantage. The ropivacaine crystal A provides sustained release of the active ingredient, resulting in postoperative pain management for up to 72 hours, approximately 7 times longer than the duration of action of conventional ropivacaine (10 hours or less). In addition, the long-lasting analgesic effect greatly reduces the amount of opioid analgesics used, resulting in a more satisfactory analgesic effect for the patient.

In addition, the methods of the present disclosure produce good analgesia, requiring fewer rescue analgesics or no opioids to manage postoperative pain. In some regimens, even multimodal analgesic therapy is not required. In some embodiments, the methods of the present disclosure produce an unexpectedly good therapeutic effect, such that only opioids are required for postoperative rescue analgesics.

### BRIEF DESCRIPTION OF THE DRAWINGS

FIG. 1: X-ray diffraction pattern of the ropivacaine crystal A

FIG. 2: Example 1 Microcrystal crystal habit diagram

FIG. 3: Time-pain intensity score at rest overlay curve

FIG. 4: Overlay plot of average plasma drug concentration-time curves of subjects in each group

### DETAILED DESCRIPTION OF THE EMBODIMENTS

### Definitions

As used herein, the term “pain” refers to a physiological and/or psychological reaction or response to a potential or actual stimulus that may result in tissue damage, injury, disease, or other condition. Types of pain include but are not limited to, acute pain, chronic pain, thermal pain, traumatic pain, chemical pain, inflammatory pain, ischemic pain, dull pain, sharp pain, stabbing pain, visceral pain, and neuropathic pain.

Unless indicated to the contrary, the compounds of the present disclosure, in addition to the specific structure of the compound, can also be read as extended to include pharmaceutically acceptable salts of the compound, stereoisomers, isotopic isomers (e.g. deuterides), solvates, hydrates, prodrugs, metabolites thereof, i.e. pharmaceutically acceptable salts of the compound, stereoisomers, isotopic isomers, solvates, hydrates, prodrugs, metabolites thereof, as well as the scope of protection of the compound.

The term “treatment” refers to medical treatment of any subject in need thereof. The treatment may be prophylactic, palliative, symptomatic, and/or curative. The duration and purpose of the treatment may vary from individual to individual, depending on the subject's state, health, and the like.

The term “treating or preventing” means attenuating, inhibiting, reversing, slowing, or stopping the progression or severity of an existing condition, disease, disorder, or symptom. Including, but not limited to, curing, alleviating, lessening, eliminating, etc. of the progression or severity of an existing disease, condition, disorder, or symptom.

Unless otherwise indicated, the quantities of various ingredients and reaction conditions used herein are to be interpreted as being “approximate” and “approximate” in any instance. Accordingly, unless indicated to the contrary, the numerical parameters set forth below and in the claims are approximations that may vary depending upon the standard error under the respective experimental conditions.

As used herein, “individual in need thereof” means a mammal, preferably a human, that is also a non-human animal, including non-human primates (e.g. monkeys, cynomolgus monkeys, etc.), companion animals (e.g. cats, dogs, etc.), farm animals (e.g. cows, sheep, pigs, horses, etc.), and rodents (e.g. rats, mice, guinea pigs, etc.) that have a condition, disease, disorder, or symptom in need of treatment or prevention.

The term “subject” refers to both humans and animals (e.g. mammals), including, but not limited to, humans, non-human primates, rodents, and the like, which will be the recipient of a particular treatment.

In general, the terms “subject” and “patient” are used interchangeably herein when referring to a human subject.

As used herein, the term “subject” and the term “individual in need thereof” have the same meaning and can be used for reference to each other.

As used herein, an “effective amount” means an amount, concentration, or dose of one or more compounds of the present disclosure, or a pharmaceutically acceptable salt thereof, that provides the desired effect (i.e. that can produce a clinically measurable difference in the condition of the individual, such as a reduction in pain perception) in the subject being diagnosed or treated after single or multiple doses are administered to the individual in need thereof. An effective amount can be readily determined by one skilled in the art by using known techniques and by observing results obtained under analogous circumstances. In determining the amount effective for an individual, a number of factors are considered including, but not limited to, the species of mammal, the size, age, general health, the particular disease or disorder involved, the severity of the disease or disorder, the response of the individual, the particular compound administered, the mode of administration, the bioavailability characteristics of the preparation administered, the dose regimen selected, the use of concomitant medication, and other relevant circumstances.

The term “therapeutically effective amount” in reference to ropivacaine or a salt thereof, e.g. ropivacaine, in the pharmaceutical compositions described herein means that the ropivacaine crystal A is released in a manner sufficient to achieve a specified level of anesthesia. The specific dose will vary depending on the particular anesthetic, as well as the age, sex, general condition of the patient, size of the patient, etc. One skilled in the art can readily take these factors into account and use them to determine a superior therapeutic concentration without undue experimentation.

The term “single injection volume” refers to the amount of the ropivacaine crystal A administered once at each site. For administration by injection, this means the amount of the pharmaceutical composition to be administered at one time, e.g. 1 mL, 2 mL, 3 mL, 4 mL, 5 mL, and 6 mL.

The term “unit dose” refers to the quantity of active ingredients contained in a unit dosage form. For example, for an injection, it typically refers to an amount of active ingredient contained per ampoule, e.g. 800 mg, 700 mg, 600 mg, 500 mg, 400 mg, 300 mg, 200 mg, etc.

The term “local infiltration” refers to: the anesthetic is injected directly into the skin and deeper tissues (e.g. subcutaneous tissue, muscular), causing a temporary loss of sensation in the nerve endings at the injection area. In the treatment of hemorrhoids, doctors usually perform local infiltration anesthesia around the anus to ensure that the treatment process is painless, especially when surgical excision of hemorrhoids or injection of sclerosing agent is performed.

The pharmaceutical compositions of the present disclosure may be administered topically or topically. As used herein, “regional” or “local” anesthesia, as opposed to general anesthesia, refers to a route of administration that permits preferential delivery of the anesthetic to a particular region of the body to be administered, e.g. near a nerve or tract of a nerve. In contrast, general anesthesia allows systemic administration, e. G. by intravenous administration. Regional or local anesthesia generally allows the administration of lower systemic concentrations, albeit elevated local concentrations, of anesthetic agents to a subject to relieve pain or lessen pain sensation in at least a portion of the subject's body.

The term “lithotomy position” refers to the position most commonly used in anal surgery. The patient is supine on the examination bed, and the buttocks are close to the bedside and put on the supporting leg stand, so as to maximize the exposure of the perineum. It is a common position for rectal and anal tube surgery and gynecological examination.

In the present disclosure, the “particle size” specifically refers to the particle size measured after the suspension has been sonicated and dispersed (the suspension particles are seen to be dispersed into single crystals under a microscope). When the suspension is not dispersed and the particle size is directly detected, the crystals have different degrees of agglomeration, and the detected particle size cannot represent the true size of the crystals.

In the present disclosure, the particle size distribution width (SPAN), which is an indicator of the crystal distribution width, is calculated by the following formula: SPAN=(D90−D10)/D50.

In the present disclosure, the term “aspect ratio” refers to the ratio of the length (longest dimension) to the width (shortest dimension perpendicular to the length) of a crystal particle, unless otherwise specified. The aspect ratio may be determined by measuring the dimensions of the crystal from microscopic images, such as optical microscopy or scanning electron microscopy (SEM) images.

As used herein, the term “habit” refers to the characteristic external shape or morphology of a crystal, which reflects the relative growth rates of different crystal faces under specific conditions. The habit of a crystal may include, but is not limited to, forms such as needle-like, plate-like, prismatic, or block-like shapes. The habit may be observed using techniques such as optical microscopy or scanning electron microscopy (SEM).

In this disclosure, the definition of therapeutic effect is to be understood in the art. In some embodiments of the present disclosure, efficacy is defined as an improvement in any of the results monitored in the studies described herein.

### EXAMPLES

### Example 1 Preparation of the Ropivacaine Crystal A

An appropriate amount of ropivacaine hydrochloride was weighed, water was added, and the mixture was stirred and dissolved at room temperature, after complete dissolution, sodium hydroxide solution was added to adjust pH>9, and then the drug solution was heated to 90° C.-100° C., sheared and dispersed while heating. After holding at 90° C. for 60 minutes, samples were taken to detect changes in the particle size and crystal habit of the single crystal during heating.

Test method for particle size: 2 ml of the drug solution was mixed with 6 ml of 0.1% Tween 80, the mixture was placed into a penicillin bottle, mixed well, dispersed with a Sonics VCX150 sonicator, the optical concentration of the dispersion was adjusted to 5%-10% to measure. The ultrasonication parameters were as follows: power: 25%, 10 s of sonication followed by 5 s of pause, cumulative sonication time: 10 min.

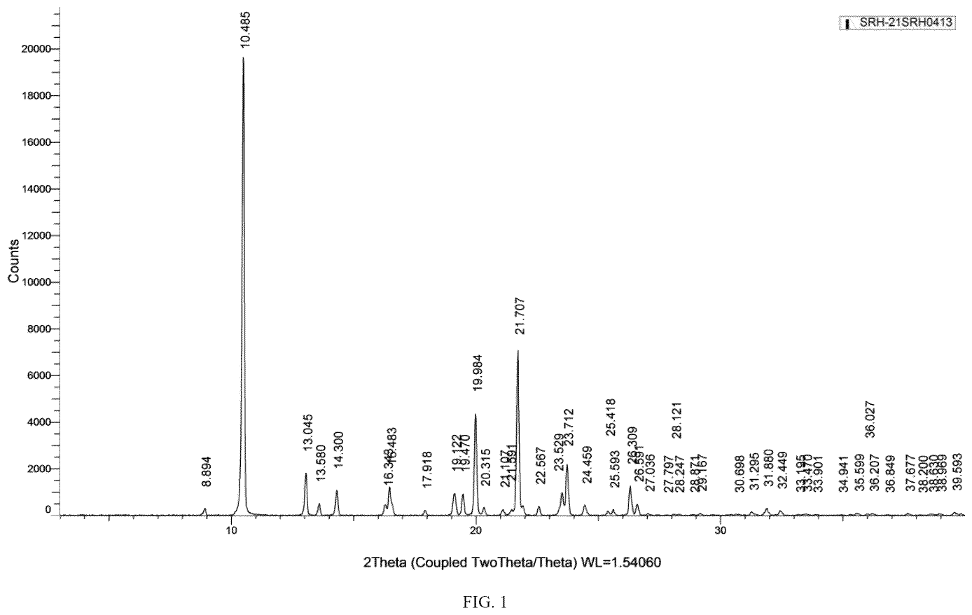

Results: As shown in FIG. 1, SPAN=1.1-1.2.

### Example 2 Analgesic Effect Trial

This trial compared the postoperative analgesic effects of a single-dose injection of the ropivacaine crystal A and ropivacaine hydrochloride in local infiltration analgesia in subjects undergoing hemorrhoidectomy.

### 1. Subjects

### 1.1 Inclusion Criteria of Subjects

The subject was able to understand the procedures and methods of this clinical trial (including the pain intensity evaluation method), and after sufficient informed consent, the subject voluntarily participated in the study and signed the informed consent form by the subject himself/herself or legal guardian.

Male or female, age ≥18 years; the body mass index (BMI) is within the range of 18.0-30.0 kg/m² (including critical value).

For subjects with degree III-IV internal or external hemorrhoids or mixed hemorrhoids combined with prolapse, Milligan-Morgan under general anesthesia is planned, and the cumulative skin incision length is ≥3 cm.

Subjects graded I-III according to the American Society of Anesthesiologists (ASA).

Female or male subjects of child-bearing age must agree to take effective contraception measures by themselves and their partners within 30 days after signing the informed consent form and after using the trial drug.

### 1.2 Exclusion Criteria of Subjects

(1) Patients who are allergic or have a history of specific reactions to amide local anesthetics, opioids, or any component of the drugs used in this trial, or allergic constitution (e.g. allergic to two or more drugs, food, or pollen).
(2) Patients with significantly prolonged QTc interval, or other risk factors (such as heart failure, hypokalemia, hypomagnesemia, bradycardia, hereditary long QT syndrome) leading to torsades de pointes (TdP), or who are taking drugs leading to prolonged QT/QTc interval.
(3) Pain associated with a medical condition or procedure not related to hemorrhoidectomy (e. g. rheumatoid arthritis, chronic neuropathic pain, concomitant vasectomy, etc.) may confound the postoperative pain evaluator.
(4) Subject with a prior history of hemorrhoidectomy.
(5) Combined with fecal incontinence, perianal abscess, anal fissure, anorectal prolapse, portal hypertension with rectal varicose veins, and inflammatory bowel disease.
(6) Patients with any one of the following clinical serious disease history or existing serious disease: \textcircled{1} respiratory diseases: such as acute or bronchial asthma, pulmonary heart disease and other chronic lung diseases, etc. or with the history of respiratory depression; \textcircled{2} neuropsychiatric diseases: such as craniocerebral injury, increased intracranial pressure, depression, anxiety, epilepsy history (including the history of hyperpyrexia convulsion in childhood), other brain diseases or mental state diseases affecting self-evaluation ability, etc.; \textcircled{3} circulatory system diseases: patients with unstable angina pectoris and/or myocardial infarction within the past 6 months, heart failure (III-IV according to the New York Heart Association [NYHA]) or poorly controlled hypertension (systolic ≥160 mmHg or diastolic ≥90 mmHg); \textcircled{4} hematological diseases: hemorrhagic disease caused by coagulation factor deficiency or other coagulation indicator disorders; \textcircled{5} digestive system diseases: such as known gastrointestinal bleeding, gastrointestinal obstruction or perforation, and other diseases which may cause risks by stimulating the gastrointestinal tract; \textcircled{6} patients with other diseases or disease histories that may significantly affect the safety evaluation or in vivo metabolism of the drug for the trial.
(7) Vomiting, or a history of severe or intractable postoperative nausea or vomiting during the screening period.
(8) the laboratory test results in the screening period meet the following requirements: \textcircled{1} glutamine-alanine aminotransferase (ALT) or glutamine-oxaloacetic aminotransferase (AST)≥2 times the upper limit of normal; \textcircled{2} blood creatinine (Cr)≥: 1.5 times of the upper limit of normal; \textcircled{3} creatinine clearance <50 mL/min (using Cockcroft-Gault formula); \textcircled{4} fasting venous blood glucose>7.8 mmol/L; \textcircled{5} prothrombin time (PT)>upper limit of normal+3 s or activated partial thrombin time (APTT)>upper limit of normal+10 s.
(9) Patients who have used the following drugs or/and treatments within a certain time before the procedure: a. used or in use of anticonvulsants, monoamine oxidase (MAO) inhibitors, tricyclic antidepressants, antipsychotics, serotonin reuptake inhibitors, norepinephrine reuptake inhibitors (SNRI), muscle relaxants, anticholinergics, etc. within 2 weeks before the procedure; b. use of opioids or opioid-like drugs within 2 weeks before the procedure; c. use of systemic steroids (except topical inhaled steroids) from within 5 half-lives before the procedure or within 10 days (whichever is longer) to 72 h after the end of administration; d. use of local anesthetics within 72 h before the procedure; e. patients who may or may plan to use analgesics other than those specified in the protocol [such as non-steroidal anti-inflammatory drugs (except low-dose aspirin for cardiac protection), oxycodone, ketorolac, ketoprofen, diclofenac or pregabalin, etc.] from 12 h before the procedure to 72 h after the end of administration; f. intravenous or oral metronidazole within 3 days before the procedure.
(10) Patients who are unable to quit smoking or alcohol withdrawal (except for therapeutic drugs containing alcohol) from 48 h before the procedure to 72 h after the end of the administration.
(11) Patients who drink more than 14 units of alcohol per week (1 unit=360 mL beer or 45 mL spirit with 40% alcohol or 150 mL wine) within 3 months (90 days) prior to screening.
(12) Patients who have undergone major surgery within 3 months (90 days) prior to screening, or who have undergone surgery that may significantly affect the in vivo process or safety evaluation of the trial drug.
(13) Patients who have participated in a drug or device clinical trial within 3 months (90 days) prior to screening and received treatment with the investigational drug or device.
(14) Patients who have a history of faint needles or faint blood, or who cannot tolerate venipuncture blood collection.
(15) Patients who have a history of drug abuse within 2 years prior to screening.
(16) Patients who have positive blood or urine pregnancy tests during the screening period, or who are breastfeeding, or who currently have no breast milk but are less than 6 months after delivery.
(17) The investigator considers that it is not suitable to participate in the trial, such as the efficacy cannot be evaluated or the expected course of treatment and follow-up are unlikely to be completed

### 2. Trial Design and Process

This trial is a phase 2, multicenter, randomized, double-blind, positive control study. A study was conducted on 24 subjects scheduled for elective hemorrhoidectomy to collect efficacy, safety, and pharmacokinetic (PK) data. The subject received a single dose of the ropivacaine crystal A for Injection 600 mg for local infiltration at the perianal site and ropivacaine hydrochloride 75 mg for local infiltration at the perianal site. Subjects were randomly assigned to the ropivacaine crystal A 600 mg intervention group and the ropivacaine hydrochloride 75 mg intervention group.

The trial period is approximately 18 days, which is divided into a screening period and an observation period, including a screening period of 2 weeks and an observation period of 4 days (post-dose analgesia observation lasts for 72 hours before leaving the group).

TABLE 1
Subjects

| | | Ropivacaine | |
|---|---|---|---|
| | Ropivacaine | hydrochloride | |
| | crystal A | injection | |
| | group | group | Total |
| Indexes | (N = 11) | (N = 12) | (N = 23) |

ASA Level, n (%)

| Missing | 0 | | 0 | | 0 | |
|---|---|---|---|---|---|---|
| Level I | 10 | (90.9%) | 11 | (91.7%) | 21 | (91.3%) |
| Level II | 1 | (9.1%) | 1 | (8.3%) | 2 | (8.7%) |

| Level III | 0 | 0 | 0 |
|---|---|---|---|
| Level IV | 0 | 0 | 0 |
| P-value | >0.9999 | | |

History of hemorrhoid diseases

| Type of disease, n (%) | | | | | | |
|---|---|---|---|---|---|---|
| Missing | 0 | | 0 | | 0 | |
| Internal hemorrhoid | 0 | | 0 | | 0 | |
| Degree III | | | | | | |
| Internal hemorrhoid | 0 | | 0 | | 0 | |
| Degree IV | | | | | | |
| External hemorrhoid | 0 | | 0 | | 0 | |
| Mixed hemorrhoids | 11 | (100%) | 12 | (100%) | 23 | (100%) |

| with prolapse | | | |
|---|---|---|---|
| Others | 0 | 0 | 0 |
| P-value | — | | |

### 2.1 Screening Period (D-14 to D-1)

From Day-14 to Day-1, after the subject voluntarily signs the written informed consent form, the inclusion and exclusion criteria shall be screened to confirm whether the subject meets the trial conditions.

### 2.2 Observation Period (D1 to D4)

Before the trial, the subjects were to be communicated with the drug administration and NRS pain intensity score training (if any) in the clinical trial. In D1, the subjects will receive hemorrhoidectomy under general anesthesia. After the operation is over (skin suture is over), a single local infiltration of the investigational drug will be carried out. Subjects will leave the group after completing the safety examination 72 h after the end of dosing.

The start and stop time of surgery, as well as other surgical details (including the length of surgical incision) shall be recorded in eCRF; subject will receive study medication unless he experiences clinically significant events (e.g. excessive bleeding, hemodynamic instability) that may destabilize the body condition or complicate the postoperative course during the procedure; the study medication was administered by local infiltration at the end of the procedure (end of skin closure). For complete details on the study medication administration technique, please refer to the administration operation manual. The start and stop time of study medication administration will be recorded in eCRF.

Note: for all efficacy, safety evaluation, and PK collection, the end time of trial drug administration will be considered 0 h, and concomitant medications used during the procedure will be recorded (note that intravenous infusion and oxygen will not be recorded unless used to treat AEs).

The subjects will be kept in the hospital for observation for at least 72 h after administration of the trial drug, and the subjects will be discharged after completion of safety examination at 72 h after the end of administration. The study procedures and evaluation are listed below.

### NRS Pain Intensity Score:

Resting and exercise pain scoring time points: within 1 h before the procedure, 2 h, 4 h, 6 h, 8 h, 12 h, 18 h, 24 h (time window of +15 min) and 36 h, 48 h, 60 h and 72 h (time window of +1 h) after the end of administration; the resting pain score is completed prior to the exercise score, and at the time of the resting pain assessment, the subject should be placed in a resting position (supine or supine) for at least 5 min.

Resting and exercise scores were performed prior to the administration of the rescue analgesics.

The pain intensity of dressing change or defecation was scored after each dressing change or defecation. The pain intensity of rest and exercise was not scored within 60 min after dressing change or defecation. The subject was administered with rescue analgesics during dressing change or defecation. The pain intensity of dressing change or defecation was evaluated only before the administration of rescue analgesics (the pain intensity of rest and exercise was not scored).

Use of rescue analgesics;
Vital signs: at 12 h, 24 h (time window: +15 min) and 48 h, 72 h (time window: +1 h) after the end of administration;
12-lead ECG: ECG monitoring within 2 h after the end of dosing and test at 72 h after the end of dosing (out of the group, without time window);
PK blood sample collection: 15 min (time window: +2 min), 1 h, 4 h, 8 h, 12 h, and 24 h (time window: ±15 min) and 48 h and 72 h (time window: ±1 h) after the end of administration; physical examination: 72 h±2 h after the end of administration;
blood routine test;
urine routine test;
blood biochemistry test;
coagulation routine test;
blood/urine pregnancy test: applicable only to females of childbearing potential;
at 72 h after the end of administration, the subject's satisfaction with analgesia;
concomitant medication records;
monitoring of safety information.

### 3. Method for Administration

In D1, the subjects receive hemorrhoidectomy under general anesthesia. After the operation is over (skin suture is over), a single local infiltration of the trial drug is carried out.

### 3.1 Drug Preparation

### Preparation of Ropivacaine Drug:

aspirating drug solution: using a 30 ml syringe, draw up 7.5 mL of ropivacaine injection from the vial;
diluting drug solution: with the same syringe, withdraw 0.9% sodium chloride injection to a total volume of 30 mL, and gently agitate the syringe by hand to ensure uniform mixing. replacing the syringe needle with an 8-gauge needle.
placing in a sterile tray for immediate use.

### Preparation of the Ropivacaine Crystal A for Injection:

preparation of sterile water for injection: drawing 4 mL of sterile water for injection into each of three 5 mL syringes;
preparation of 0.9% sodium chloride injection: drawing 18 mL of 0.9% sodium chloride injection into a single 30 ml syringe;
trial drug re-suspension: taking 3 bottles of trial drug, slowly injecting three 4 mL vials of sterile water for injection along the inner wall of the bottle, and gently shaking to fully dissolve the drug;
aspirating drug solution: taking a 30-mL syringe and aspirating all the re-suspension in step [3], gently shaking it manually, and mixing it well;
rinsing the bottle: slowly injecting 18 mL of 0.9% sodium chloride injection along the inner walls of the three bottles, gently shaking to rinse the residual drug solution completely, completely drawing the rinse solution into the syringe of step [4], gently shaking it manually, and mixing it well. replace the syringe needle with an 8-gauge needle.
placing in a sterile tray for immediate use.

### Key Points of Preparation:

during the operation, the principles of aseptic operation and checking system shall be strictly implemented, and the used disposable syringes, 0.9% sodium chloride injection, and sterile water for injection shall be subject to the external packaging, abandoned drugs, and other medical wastes shall be treated in strict accordance with the relevant provisions on medical wastes of the Study Site. The used trial drug package and packaging box shall be recovered by the drug administrator, and stored in a specific area, and relevant information shall be registered and signed for confirmation.

The review personnel review the whole process of preparation, record the information related to preparation in the process of preparation (including but not limited to preparation start/end time, configuration environment temperature, etc.), and the operator confirms and signs the preparation information after the operation.

The drug for the trial is prepared at room temperature, and it is recommended that the investigational drug is used within 2 hours after the end of preparation.

Syringes are specially designed for human and drug use, and cannot be used in tandem.

In order to reduce the operation deviation in the drug preparation process as far as possible, 1-2 persons shall be responsible for drug preparation as far as possible during the trial, so as to ensure the consistency of operation.

During the preparation process, the mixing operation should not be shaken vigorously to avoid the generation of bubbles in the drug solution.

### 3.2 Drug Administration Procedure

3.2.1 air draining: the air in the syringe was drained until a droplet appeared on the tip of the needle, and the actual volume of drug solution to be used in each syringe was recorded.
3.2.2 Administration by local infiltration injection:
a) Needle insertion site: the needle was inserted at 3, 6, 9, and 12 o'clock positions of the lithotomy position, about 0.5-3 cm away from the anal verge.
b) Needle insertion angle:
i. The needle was inserted vertically deep into the ischiorectal fossa at 12 o'clock and 6 o'clock.
ii. The needle was inserted vertically at 3 o'clock, 9 o'clock position, 45° downward, and 45° upward, needle depth greater than 3 cm.
3.2.3 Administration volume (30 mL):
a) 4 mL of drug solution was administered for each injection at 12 o'clock and 6 o'clock position by withdrawing the needle.
b) 2 mL of drug solution was administered in each direction for each injection at 3 o'clock and 9 o'clock position by withdrawing the needle.
3.2.4 The surgical wound was locally infiltrated with about 10 ml of the drug solution.
3.2.5 Upon completion of the injection, the start and end of administration time shall be recorded, and it is recommended to complete the administration within about 5 min

### 4. Concomitant Therapy During the Procedure

The opioids such as pethidine, fentanyl, sufentanil, alfentanil, remifentanil, and methadone should not be used during the trial.

Acetaminophen should not be used during the trial. Aspirin should not be used during the trial.

NSAIDs such as diclofenac sodium, parecoxib sodium, and ibuprofen should not be used during the trial.

Local anesthetics such as procaine, chloroprocaine, lidocaine, and bupivacaine should not be used during the trial.

Glucocorticoids such as dexamethasone and methylprednisolone should not be used during the trial.

Antiepileptic drugs such as carbamazepine, oxcarbazepine, sodium valproate, pregabalin and gabapentin should not be used during the trial.

Anxiolytics such as chlordiazepoxide and diazepam should not be used during the trial.

Antidepressants such as SSRIs, SNRIs, SARIs, NDRIs, TCAs, MAOIs, NASSA, and NRI should not be used during the trial.

Class III antiarrhythmic drugs should not be used during the trial.

Potent CYP1A2 inhibitors, including ciprofloxacin and fluvoxamine, should not be used during the trial.

CYP1A2 highly sensitive substrates including alosetron, caffeine, duloxetine, melatonin, ramelteon, tasimetron, and tizanidine should not be used during the trial.

The use of potent CYP3A4 inhibitors such as ceritinib, clarithromycin, combiastat, entegravir combined and ritonavir, idelaris, indinavir combined and ritonavir, itraconazole, ketoconazole, lopinavir combined and ritonavir, nefazodone, nelfinavir, palirivir combined and ritonavir combined and (obitavir and/or dalatavir), posaconazole, ritonavir, saquinavir combined and ritonavir, telithromycin, tipranavir combined and ritonavir, voriconazole should not be used during the trial. The use of CYP3A4 highly sensitive substrates should not be used during the study included alfentanil, avanafil, budesonide, buspirone, conivaptan, darunacin, darunavir, dasatinib, dronedarone, eletriptan, eplerenone, everolimus, felodipine, ibrutinib, indinavir, isaconazole, ivabradine, laborexan, lometapide, lovastatin, lurasidone, malaviroc, midazolam, motozantinib, naloxol, nisoldipine, quetiapine, saquinavir, sildenafil, simvastatin, sirolimus, tacrolimus, ticagrelor, tipranavir, tolvaptan, triazolam, vardenafil, vinetoc.

Potent CYP3A4 inducers, including apalutamide, carbamazepine, enzalutamide, avoneb, lumacor, mitotane, phenytoin, rifampicin, st. John's wort, should not be used during the trial.

Physical therapy which may have an analgesic effect during the trial should not be used during the trial.

Anti-emetic agents such as 5-HT3 agents that potentially prolong the QTc interval should not be used during the trial.

Prophylactic use of anti-emetics should not be used during the trial.

Rescue analgesics should not be used to prevent pain, but only for postoperative rescue analgesia, and should be provided at any time after the initial dose of the trial drug, so as to keep subjects comfortable. Morphine 2 mg (first time) was intravenously injected as postoperative rescue analgesia, followed by a slow bolus injection followed by an on-demand supplement (not more than 10 mg on-demand within 2 h). Other analgesics, including non-steroidal anti-inflammatory drugs, are not allowed within 72 h after administration.

When the subject has a feeling of nausea/vomiting or the subject's feeling of nausea requires the use of antiemetic drugs, metoclopramide Injection, aprepitant Injection, or Fosapitant Dimeglumine for Injection may be used, and antiemetic drugs (such as Ondansetron and Domperidone) causing the prolongation of QTc interval are prohibited. The use of antiemetic drugs follows the usage and dosage specified in the instructions for use, but should not be used prophylactically (i.e. the use of prophylactic antiemetic drugs is prohibited without signs/symptoms of nausea/vomiting or regardless of the subject's needs).

### 5. Trial Results

### 5.1 Efficacy Evaluation

Pharmacodynamic assessments included pain intensity scores for the surgical anal site using a numerical rating scale (NRS), and the time to first opioid use after surgery.

Wherein, NRS pain intensity score:

Resting and exercise pain scoring time points: within 1 h before the procedure, 2 h, 4 h, 6 h, 8 h, 12 h, 18 h, 24 h (time window: +15 min) and 36 h, 48 h, 60 h and 72 h (time window: +1 h) after the end of administration. The rest pain score was completed prior to the exercise score. For the assessment of resting pain, subjects should be placed in a resting position (supine or supine) for at least 5 min.

Resting and exercise scores were performed prior to the administration of the rescue analgesics.

After each dressing change or defecation, the pain intensity of dressing change or defecation was scored, and the pain intensity of resting and exercise was not scored within 60 min after dressing change or defecation. The subject was given remedial analgesia during dressing change or defecation, and only the pain intensity during dressing change or defecation was assessed before the administration of rescue analgesics (no need for resting and exercise scores).

The time points for PK collection are: within 1 h before the procedure (blank sample), 15 min (time window: +2 min), 1 h, 4 h, 8 h, 12 h, 24 h (time window: +15 min), 48 h and 72 h (time window: +1 h) after the end of administration.

Satisfaction score for analgesia was: before discharge, the subjects scored satisfaction with analgesia within 72 h after administration, using a five-grade scoring system (very satisfied=4, satisfied=3, general=2, dissatisfied=1, very dissatisfied=0).

The area under the pain intensity score-time curve (AUC_{0-72 h}) at rest-time of the primary efficacy endpoint was analyzed by analysis of covariance (ANCOVA) to compare the differences of the primary efficacy indicators between the trial drug and the control drug; the main efficacy indicators will be analyzed by ANCOVA model with treatment group as main effect and baseline NRS value as covariate. At the bilateral 5% significance level, the differences in the main efficacy indicators between the trial drug and the control drug will be compared, and the least squares mean, standard deviation, standard error and 95% CI of intra-group and inter-group differences between AUC_{0-72 h} will be calculated respectively.

For the secondary efficacy endpoint, the pain intensity score-area under the curve was calculated using the Linear Trapezoidal Linear Interpolation method. For continuous variables, based on their definitions, the analysis was performed using the same method as the primary analysis method for the primary estimation objective. For dichotomous variables, the proportion of subjects within the group and the proportion difference between groups reaching the relevant endpoints and their 95% CI were calculated, respectively, and the chi-square test or Fisher exact probability method was used for analysis and the P value was reported. Wilcoxon rank-sum test was used for grade data analysis and the p-value was reported.

The results are shown in Tables 2-4.

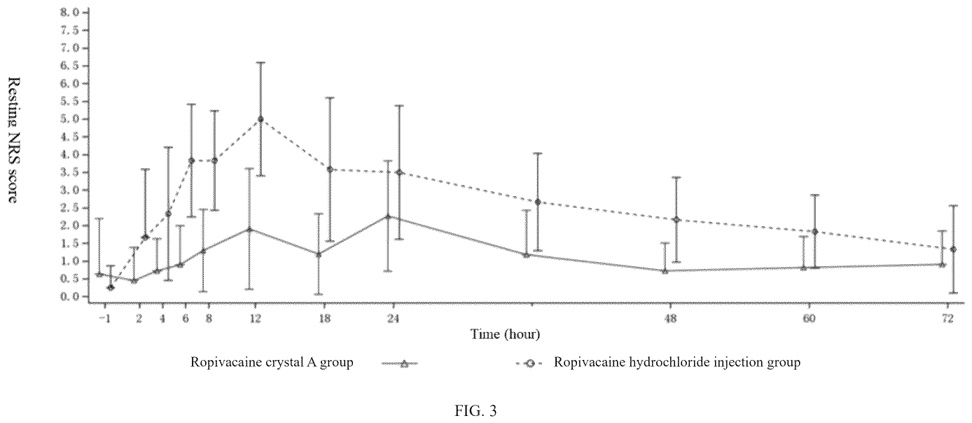

The time-to-rest pain intensity score overlay curve is shown in FIG. 3. In FIG. 3, the solid line represents the ropivacaine crystal group A, and the dotted line represents the ropivacaine hydrochloride injection group.

TABLE 2
Comparison of the area under the pain intensity score-
time curve at rest (AUC_{0-72 h}) - analysis of covariance

| | | | | Difference between groups^{1′ 2} | | |
|---|---|---|---|---|---|---|
| | | Mean | | Least squares mean of | | |
| | | (standard | | difference between groups | | |
| Treatment group | n | error) | | (standard error) | 95% CI | P-value |
| Ropivacaine crystal | 11 | 83.2 | (16.02) | −109.1 (22.33) | −155.66, | <0.0001 |
| A group | | | | | −62.49 | |
| Ropivacaine | 12 | 192.3 | (15.33) | | | |

hydrochloride
injection group

Note:
¹The area under the pain intensity score-time curve at rest (AUC_{0-72 hr}) was compared between groups using analysis of variance (ANCOVA). The ANCOVA model will use area under the curve of subject 0-72 h resting pain intensity score (AUC_{0-72 h}) as the dependent variable, group as the independent variable, and include the baseline resting pain score value as the covariate.
²Compare between groups (the ropivacaine crystal A 600 mg group Vs. ropivacaine hydrochloride injection group), and calculate the difference between groups.

The main analysis results showed that the mean values of the area under the pain intensity score-time curve AUC_{0-72 h} at rest within 72 h in the ropivacaine crystal A 600 mg group (N=11) and ropivacaine hydrochloride injection group (N=12) were 83.2±16.02 and 192.3±15.33, respectively. The ropivacaine crystal A 600 mg group was significantly superior to the ropivacaine hydrochloride injection group (P<0.0001).

For the secondary efficacy indicators, based on m-ITT set, the analysis results of the area under the pain intensity score-time curve at rest at 0-24 h, 0-48 h and 24-72 h after administration and the area under the pain intensity score-time curve in exercise at 0-24 h, 0-48 h, 0-72 h and 24-72 h after administration of the secondary efficacy indicators showed the same trend as that of the primary efficacy indicators, and the ropivacaine crystal A 600 mg group had significant superiority in postoperative analgesia compared with the ropivacaine hydrochloride injection group.

TABLE 3
Analysis of the area under the pain intensity score-
time curve for secondary efficacy indicators

| | | | | Difference between groups^{1′ 2} | | | |
|---|---|---|---|---|---|---|---|
| | | Mean | | Least squares mean of | | | |
| Treatment time | | (standard | | difference between groups | | | |
| Treatment group | n | error) | | (standard error) | | 95% CI | P-value |
| 0-24 h | | | | | | | |
| Ropivacaine crystal A | 11 | 30.5 | (5.43) | −53.6 | (10.12) | −74.69, | <0.0001 |
| 600 mg group | | | | | | −32.47 | |
| Ropivacaine | 12 | 84.2 | (7.87) | | | | |
| hydrochloride | | | | | | | |
| injection group | | | | | | | |
| 0-48 h | | | | | | | |
| Ropivacaine crystal A | 11 | 62.6 | (9.66) | −86.5 | (18.04) | −124.10, | 0.0001 |
| 600 mg group | | | | | | −48.84 | |
| Ropivacaine | 12 | 150.2 | (14.09) | | | | |
| hydrochloride | | | | | | | |
| injection group | | | | | | | |
| 24-72 h | | | | | | | |
| Ropivacaine crystal A | 11 | 51.8 | (7.16) | −55.5 | (15.47) | -87.76, | 0.0018 |

| 600 mg group | | | | -23.23 |
|---|---|---|---|---|
| Ropivacaine | 12 | 109.0 | (12.79) | |

hydrochloride
injection group

Note:
¹The area under the pain intensity score-time curve at rest-time between groups was compared using analysis of variance (ANCOVA). The ANCOVA model will take the area under the curve of the corresponding resting pain intensity score (AUC) of subjects as the dependent variable, group as the independent variable, and include the baseline resting pain score value as the covariate;
²Comparison between groups (CZ1S 600 mg group vs. ropivacaine hydrochloride injection group), and the difference between groups was calculated.

The mean of the area under the pain intensity score-time curve AUC_{0-24 h} at rest within 0-24 h after administration in the ropivacaine crystal A 600 mg group (N=11) and ropivacaine hydrochloride injection group (N=12) were 30.5±5.43 and 84.2±7.87, respectively. The ropivacaine crystal A 600 mg group had significant superiority compared to the ropivacaine hydrochloride injection group (P<0.0001).

The mean of the area under the pain intensity score-time curve AUC_{0-48 h} at rest within 0-48 h after administration in the ropivacaine crystal A 600 mg group (N=11) and ropivacaine hydrochloride injection group (N=12) were 62.6±9.66 and 150.2±14.09, respectively. The ropivacaine crystal A 600 mg group had significant superiority compared to the ropivacaine hydrochloride injection group (P=0.0001).

The mean of the area under the pain intensity score-time curve AUC_{24-72 h} at rest within 24-72 h after administration in the ropivacaine crystal A 600 mg group (N=11) and ropivacaine hydrochloride injection group (N=12) were 51.8-7.16 and 109.0±12.79, respectively. The ropivacaine crystal A 600 mg group had significant superiority compared to the ropivacaine hydrochloride injection group (P=0.0018).

TABLE 4
Inter-group comparison of the rates of non-use of rescue analgesic
drugs within 0-24 h, 24 h-72 h, and 0-72 h after administration

| | | | | Ropivacaine | |
|---|---|---|---|---|---|
| | | Ropivacaine | | hydrochloride | |
| | | crystal A 600 | | injection | |
| Treatment | | mg group | | group | |
| time | Statistics | (N = 11) | | (N = 12) | |
| 0-24 h | No usage, n (%) | 9 | (81.8%) | 3 | (25.0%) |
| | 95% CI¹ | 48.22, 97.72 | | 5.49, 57.19 | |
| | Rate difference | 56.82 | (15.97, 77.54) | — | (—, —) |
| | and 95% CI² | | | | |
| | P-Value² | 0.0123 | | | |
| 24 h-72 h | No usage, n (%) | 10 | (90.9%) | 10 | (83.3%) |
| | 95% CI¹ | 58.72, 99.77 | | 51.59, 97.91 | |
| | Rate difference | 7.58 | (−23.47, 36.69) | — | (—, —) |
| | and 95% CI² | | | | |
| | P-Value² | 1.0000 | | | |
| 0-72 h | No usage, n (%) | 9 | (81.8%) | 3 | (25.0%) |
| | 95% CI¹ | 48.22, 97.72 | | 5.49, 57.19 | |
| | Rate difference | 56.82 | (15.97, 77.54) | — | (—, —) |

| | |
|---|---|
| and 95% CI² | |
| P-Value² | 0.0123 |

Note:
¹The 95% confidence interval was calculated using the Clopper Pearson method for the proportion of patients who did not receive remedial pain medication;
²The 95% confidence interval of rate difference was estimated by the Newcombe method; fisher exact probability method was used to compare between the two groups (the ropivacaine crystal A 600 mg group vs. Ropivacaine hydrochloride injection group;), the difference between groups was calculated;
the proportion of subjects in the ropivacaine crystal A 600 mg group (N = 11) who did not use opioids at 24 h after administration was 81.8%, and the proportion of subjects in ropivacaine injection group who did not use opioids at 24 h after administration was 25.0%. The ropivacaine crystal A 600 mg group was significantly superior to ropivacaine hydrochloride injection group (P = 0.0123).
The proportion of subjects who did not use opioids at 72 h after administration in the ropivacaine crystal A 600 mg group (N = 11) was 81.8%, and the proportion of subjects who did not use opioids at 72 h after administration in ropivacaine hydrochloride injection group was 25.0%. The ropivacaine crystal A 600 mg group was significantly superior to the ropivacaine hydrochloride injection group (P = 0.0123).

Analysis of results: the proportion of subjects who did not use rescue analgesic drugs was the same in each group from 0-24 h after administration and from 0-72 h after administration, and the ropivacaine crystal A 600 mg group was significantly higher than the ropivacaine hydrochloride injection group (81.8% vs 25.0%; P=0.0123); the proportion of subjects who did not use rescue analgesic drugs was similar between the two groups from 24-72 h after administration, and the difference was not statistically significant (90.9% vs 83.3%; P=1.0000), it was considered that the pain level was gradually decreased with the absorption of the pain-causing medium, inflammatory medium and recovery of tissue injury after surgery, and the pain level was decreased to the tolerable range of the subject after administration for 24 h.

### Example 3 Pharmacokinetic Trial

Blood samples were collected for pharmacokinetic analysis (area under the curve, maximum decreased concentration, time of maximum plasma concentration, apparent terminal elimination half-life, apparent clearance rate, apparent volume of distribution).

Key PK data point arrangements: a) The PK parameters were calculated according to the actual sampling time. b) All plasma drug concentration values below the lower limit of quantitation were treated as “0” before Tmax and as missing after Tmax when PK analysis was performed. For descriptive statistical analysis, however, treatment was performed at 0, and the number of BQL at each time point was noted. Linear Trapezoidal Linear Interpolation in the non-compartmental model method (NCA module) calculated the single-dose parameters according to the actual blood collection time.

Analysis of plasma drug concentration-time (c-t) data: Based on PKCS analysis, individual and mean c-t curves and semilogarithmic c-t curves were drawn respectively; the mean, standard deviation, median, maximum, minimum, and coefficient of variation of drug concentration at each time point were analyzed.

Analysis of PK parameters: pharmacokinetic parameters were calculated for each subject from non-compartmental models based on PKPS analysis. At the same time, the arithmetic mean, standard deviation, coefficient of variation, quartile, maximum value, minimum value, geometric mean, and geometric coefficient of variation of each parameter were calculated.

Inter-group comparison: based on PKPS analysis, PK parameters were transformed by natural logarithm and analyzed by variance. The group was taken as a fixed effect. The 90% confidence interval of the geometric mean ratio (trial drug/positive drug) of PK parameters was calculated.

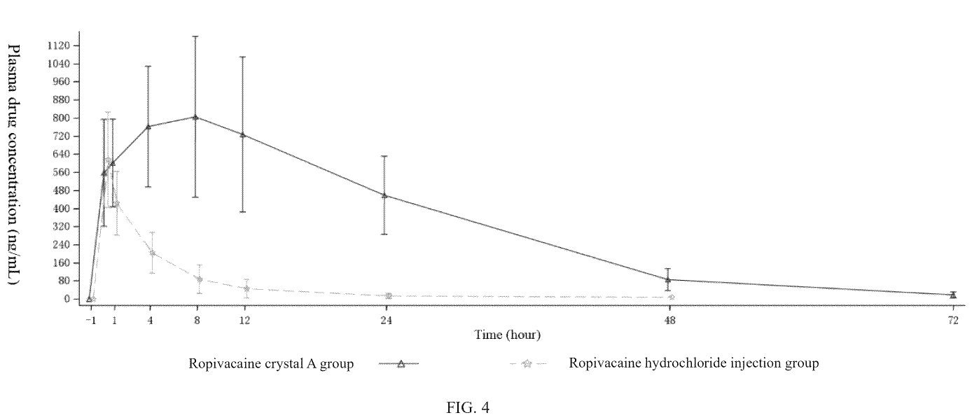

The results are shown in Tables 5 to 7, and FIG. 4.

TABLE 5
Summary description of plasma drug concentration of ropivacaine hydrochloride group

| | Plasma drug concentration (ng/mL) at each planned sampling time point (h) | | | | | | | | |
|---|---|---|---|---|---|---|---|---|---|
| | | Post- | Post- | Post- | Post- | Post- | Post- | Post- | Post- |
| | Pre- | admin- | admin- | admin- | admin- | admin- | admin- | admin- | admin- |
| | procedure | istration | istration | istration | istration | istration | istration | istration | istration |
| | Within 1 h | 15 min | 1 h | 4 h | 8 h | 12 h | 24 h | 48 h | 72 h |
| N | 12 | 11 | 12 | 12 | 12 | 11 | 6 | 1 | 0 |
| BQL number | 12 | 0 | 0 | 0 | 0 | 1 | 6 | 11 | 11 |
| Mean | 0 | 609.000 | 423.833 | 205.200 | 88.353 | 46.645 | 14.308 | 8.750 | NC |
| SD | 0 | 219.9691 | 140.9577 | 90.5920 | 63.1340 | 40.9539 | 11.7794 | NC | NC |
| CV (%) | NC | 36.12 | 33.26 | 44.15 | 71.46 | 87.80 | 82.33 | NC | NC |
| Max | 0 | 1110 | 629 | 372 | 249 | 147 | 34.6 | 8.75 | NC |
| Min | 0 | 307 | 206 | 28.4 | 3.83 | 13.2 | 2.45 | 8.75 | NC |
| Median | 0 | 575.000 | 409.500 | 204.000 | 67.700 | 28.100 | 11.950 | 8.750 | NC |
| Q1 | 0 | 514.000 | 328.500 | 152.500 | 50.950 | 19.100 | 4.600 | 8.750 | NC |
| Q3 | 0 | 735.000 | 556.000 | 263.500 | 117.000 | 72.000 | 20.300 | 8.750 | NC |
| GeoMean | NC | 573.821 | 400.069 | 177.945 | 64.194 | 34.508 | 10.206 | 8.750 | NC |
| GeoSD | NC | 1.4439 | 1.4455 | 1.9356 | 2.8065 | 2.2114 | 2.6326 | NC | NC |
| GeoCV (%) | NC | 38.01 | 38.13 | 73.94 | 137.86 | 93.67 | 124.59 | NC | NC |

TABLE 6
Summary and description of plasma drug concentration of the ropivacaine crystal A for injection 600 mg group

| | Plasma drug concentration (ng/mL) at each planned sampling time point (h) | | | | | | | | |
|---|---|---|---|---|---|---|---|---|---|
| | | 15 min | Post- | Post- | Post- | Post- | Post- | Post- | Post- |
| | Pre- | after | admin- | admin- | admin- | admin- | admin- | admin- | admin- |
| | procedure | admin- | istration | istration | istration | istration | istration | istration | istration |
| | Within 1 h | istration | 1 h | 4 h | 8 h | 12 h | 24 h | 48 h | 72 h |
| N | 11 | 11 | 11 | 11 | 11 | 11 | 11 | 11 | 10 |
| BQL number | 11 | 0 | 0 | 0 | 0 | 0 | 0 | 0 | 1 |
| Mean | 0 | 558.182 | 601.636 | 762.636 | 805.727 | 727.636 | 459.182 | 86.482 | 18.858 |
| SD | 0 | 236.1550 | 193.8264 | 266.6752 | 355.5489 | 342.6988 | 172.6539 | 48.8975 | 13.4075 |
| CV (%) | NC | 42.31 | 32.22 | 34.97 | 44.13 | 47.10 | 37.60 | 56.54 | 71.10 |
| Max | 0 | 921 | 913 | 1250 | 1570 | 1280 | 870 | 193 | 51.3 |
| Min | 0 | 164 | 234 | 337 | 444 | 351 | 255 | 16.4 | 5.23 |
| Median | 0 | 579.000 | 648.000 | 775.000 | 715.000 | 645.000 | 439.000 | 77.700 | 15.700 |
| Q1 | 0 | 442.000 | 511.000 | 578.000 | 549.000 | 436.000 | 312.000 | 56.900 | 10.100 |
| Q3 | 0 | 730.000 | 703.000 | 889.000 | 955.000 | 1200.000 | 556.000 | 95.600 | 22.600 |
| GeoMean | NC | 497.144 | 565.060 | 717.262 | 745.578 | 661.566 | 433.255 | 73.781 | 15.304 |
| GeoSD | NC | 1.7491 | 1.4951 | 1.4627 | 1.4947 | 1.5711 | 1.4219 | 1.8906 | 1.9950 |
| GeoCV (%) | NC | 60.58 | 41.90 | 39.45 | 41.87 | 47.58 | 36.32 | 70.73 | 78.18 |

TABLE 7
Summary of pharmacokinetic parameters

| | | Ropivacaine | Ropivacaine |
|---|---|---|---|
| | | crystal A | hydrochloride |
| Pharmacokinetic | | 600 mg group | injection group |
| parameters | Statistics | N = 11 | N = 12 |
| Tₘₐₓ (h) | Median (Min, max) | 7.88 (0.37, 12.2) | 0.33 (0.28, 0.4) |
| Cₘₐₓ (ng/mL) | Mean ± SD | 895.82 ± 349.02 | 615.83 ± 211.06 |
| AUC₀₋ₜ (h*ng/mL) | Mean ± SD | 23661.51 ± 8048.99 | 2522.72 ± 1059.19 |
| AUC_{0-∞} (h*ng/mL) | Mean ± SD | 23974.82 ± 7976.08 | 2598.42 ± 1087.87 |
| t_{1/2} (h) | Mean ± SD | 10.13 ± 2.98 | 3.88 ± 2.53 |

Pharmacokinetic analysis results showed that, based on the PKPS set, the pharmacokinetic parameters Tmax of ropivacaine in the ropivacaine crystal A 600 mg group (N=11) and ropivacaine hydrochloride injection group (N=12) were 7.88 (0.37, 12.2) h and 0.33 (0.28, 0.4) h, respectively, and Tmax of the ropivacaine crystal A 600 mg group was significantly longer than that of ropivacaine hydrochloride injection group (P=0.0002). Cₘₐₓ was 895.82±349.02 ng/ml and 615.83±211.06 ng/ml, AUC₀₋ₜ was 23661.51±8048.99 h*ng/ml and 2522.72±1059.19 h*ng/mL, AUC_{0-∞} was 23974.82±7976.08 h*ng/ml and 2598.42±1087.87 h*ng/ml, t_{1/2} was 10.13±2.98 h and 3.88±2.53 h, respectively. Compared with ropivacaine hydrochloride injection group, Tmax was significantly increased in the ropivacaine crystal A 600 mg group, and Cₘₐₓ was within the safety window, suggesting that the ropivacaine crystal A 600 mg could slowly release ropivacaine at a non-constant rate after local infiltration injection, providing a stable and sustained plasma drug concentration.

### Example 4 Safety Trial

This trial evaluated the safety of the ropivacaine crystal A for Injection and ropivacaine hydrochloride. Safety endpoints included the occurrence of AEs and SAEs from the start of dosing to 72 h post-procedure.

Analysis based on Safety Data Set (SS); adverse events recorded in this trial will be coded according to MedDRA (version V26.1 or above). To summarize the adverse events/adverse reactions, adverse events/adverse reactions during treatment, serious adverse events/adverse reactions during treatment, the number of cases and times of adverse events/adverse reactions leading to drop-out during treatment, and calculate the incidence rate; the number, cases, and percentage of AEs were summarized by treatment group according to SOC, PT and/or CTCAE classification.

Summarize and describe the laboratory test results of blood routine, urine routine, blood biochemistry, and coagulation routine in each treatment group at each scheduled visit as well as the changes from baseline. The change of clinical significance evaluation results of laboratory tests at each scheduled visit from baseline will be summarized in a cross-table for each treatment group. All the test results and abnormal test results of blood routine, urine routine, blood biochemistry, and coagulation routine will be listed according to the subjects.

To summarize the changes of overall clinical significance evaluation results of ECG relative to baseline in each treatment group at each scheduled visit in the form of cross-table; duration analysis of measured value and change value from baseline before and after treatment; all the ECG test results and abnormal test results will be tabulated according to subjects.

Cross-table of clinical judgment and detailed list of test results before and after treatment.

There was no death case in this trial, and the subject had good tolerance to ropivacaine crystal A for injection.

The AE rates in the ropivacaine crystal A 600 mg group (N=11) and ropivacaine hydrochloride injection group (N=12) were 4 (36.4%) vs 6 (50.0%), respectively, and the ADR rates were 2 (18.2%) vs 2 (16.7%), respectively. There were no grade ≥3 AEs, SAEs, adverse events leading to drop-off, or death, no unexpected adverse events, and no systemic toxic reactions of local anesthetics occurred.

The incidence rate of AEs in ropivacaine crystal A 600 mg group (N=11) and ropivacaine hydrochloride injection group (N=12) was 4 (36.4%) vs 6 (50.0%), respectively, according to PT classification: urinary retention 2 (18.2%) vs 4 (33.3%), fever 1 (9.1%) vs 1 (8.3%), nausea 1 (9.1%) vs 1 (8.3%), vomiting 0 vs 1 (8.3%), gastrointestinal pain 0 vs 1 (8.3%), hypoalbuminemia 0 vs 1 (8.3%), postoperative wound infection 0 vs 1 (8.3%), lymphocyte count decreased 0 vs 1 (8.3%), platelet count decreased by 0 vs 1 (8.3%), incision site bleeding by 1 (9.1%) vs 0, anemia by 0 vs 1 (8.3%). Except for 1 case of postoperative wound infection in ropivacaine injection group, which was improved/alleviated, the other AEs were recovered/resolved.

The incidence rate of ADR in ropivacaine crystal A 600 mg group (N=11) and ropivacaine hydrochloride injection group (N=12) was 2 (18.2%) vs 2 (16.7%), respectively, according to PT classification: fever 1 (9.1%) vs 1 (8.3%), nausea 1 (9.1%) vs 1 (8.3%), vomiting 0 vs 1 (8.3%), gastrointestinal pain 0 vs 1 (8.3%), hypoalbuminemia 0 vs 1 (8.3%), lymphocyte count decreased 0 vs 1 (8.3%), platelet count decreased 0 vs 1 (8.3%), anemia 0 vs 1 (8.3%).

In conclusion, the local infiltration of the ropivacaine crystal A for injection of 600 mg was safe for postoperative analgesia after hemorrhoidectomy.

While embodiments have been described above, it will be apparent to those skilled in the art that modifications can be made without departing from the disclosure. Such modifications are to be considered as possible variations included within the scope of the present disclosure.

What is claimed is:

1. A method for preventing or treating postoperative pain, comprising administering to an individual in need thereof a pharmaceutical composition comprising ropivacaine crystal A.

2. The method according to claim 1, wherein the ropivacaine crystal A has an aspect ratio of 1:(1-2).

3. The method according to claim 1, wherein at least 80%, at least 85%, at least 90%, and at least 95% of crystals in the ropivacaine crystal A have an aspect ratio of 1:(1-2).

4. The method according to claim 1, wherein the ropivacaine crystal A has a D₅₀ of 5-10 μm.

5. The method according to claim 1, wherein the ropivacaine crystal A has a crystal particle size distribution width value of 1-2.

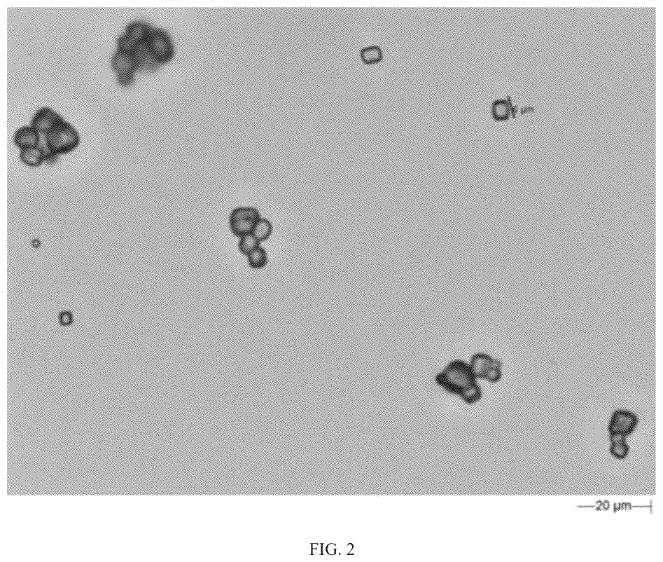

6. The method according to claim 1, wherein the ropivacaine crystal A has a particle morphology as shown in FIG. 2.

7. The method according to claim 1, wherein the ropivacaine crystal A has an XRPD diffraction pattern as shown in FIG. 1.

8. The method according to claim 1, wherein the postoperative pain comprises post-hemorrhoidectomy pain.

9. The method according to claim 1, wherein the postoperative pain comprises traumatic pain, chemical pain, neuropathic pain, and inflammatory pain.

10. The method according to claim 1, wherein the preventing or treating comprises curing, alleviating, lessening, or eliminating a progression or severity of a disease, condition, disorder, or symptom.

11. The method according to claim 1, wherein the ropivacaine crystal A is administered to the individual in need thereof by injection.

12. The method according to claim 1, wherein the ropivacaine crystal A is administered to the individual in need thereof by perianal injection, and the perianal injection comprises local infiltration.

13. The method according to claim 1, comprising:
selecting an entry point of an injection needle at a body site of the individual in need thereof, inserting the injection needle into the individual in need thereof at the entry point, and topically administering the ropivacaine crystal A to a body of the individual in need thereof via the injection needle.

14. The method according to claim 13, comprising:
selecting the entry point of the injection needle around an anus of the individual in need thereof;
selecting at least 4 sites for the entry point of the injection needle;
inserting the injection needle into the body of the individual in need thereof from the at least 4 sites; and
topically injecting the pharmaceutical composition around the anus of the individual in need thereof via the injection needle.

15. The method according to claim 14, wherein a single injection volume of the pharmaceutical composition is less than 6 mL.

16. The method according to claim 14, wherein a total amount of the pharmaceutical composition is less than 30 mL.

17. The method according to claim 14, wherein a dose of the ropivacaine crystal A in the pharmaceutical composition is 200 mg-800 mg.

18. The method according to claim 1, wherein the method further comprises co-administering the ropivacaine crystal A to the individual in need thereof with other anesthetics, an anesthesia adjuvant, or other analgesics.

19. The method according to claim 18, wherein the other anesthetics comprise general anesthetics and local anesthetics, the general anesthetics comprises etomidate, ketamine, ciprofol, propofol, and thiopental sodium; and the local anesthetics are selected from benzocaine, chloroprocaine, cocaine, cyclomethycaine, dimethocaine, propoxycaine, procaine, amethocaine, articaine, bupivacaine, etidocaine, levobupivacaine, lidocaine, mepivacaine, and trimecaine;
and/or the anesthesia adjuvant comprises midazolam and diazepam;
and/or the other analgesics comprise opioid analgesics, antipyretic analgesic anti-inflammatory drugs, and adjuvant analgesics;
wherein the opioid analgesics are selected from hydrocodone, dextropropoxyphene, codeine, dihydrocodeine, tramadol, morphine, oxycodone, hydromorphone, fentanyl, sufentanil, remifentanil, buprenorphine, pethidine (dolantin), nalbuphine, methadone, levorphanol, and pentazocine; the antipyretic analgesic anti-inflammatory drugs are selected from: acetaminophen and NSAIDs; wherein the NSAIDs are selective COX-2 inhibitors comprising celecoxib, or non-selective inhibitors comprising ibuprofen, aspirin, diclofenac, and naproxen; the adjuvant analgesics are selected from: anticonvulsants comprising gabapentin, tricyclic antidepressants comprising amitriptyline, psychotropic drugs, NMDA receptor antagonists comprising ketamine, α₂-adrenergic receptor agonists comprising dexmedetomidine, and glucocorticoids comprising dexamethasone.

20. The method according to claim 1, wherein the ropivacaine crystal A is not used in combination with other anesthetics or analgesics.
